(19) United States
(12) Patent Application Publication
(10) Pub. No.: US 2018/0116660 A1
(43) Pub. Date: May 3, 2018

(54) SURGICAL NEEDLE APPARATUS FOR RELOCATING ORBITAL FAT

(71) Applicants: Cheol Ho CHANG, Seoul (KR); Myung Kyu CHA, Seoul (KR)

(72) Inventors: Cheol Ho CHANG, Seoul (KR); Myung Kyu CHA, Seoul (KR)

(21) Appl. No.: 15/394,002

(22) Filed: Dec. 29, 2016

(30) Foreign Application Priority Data
Oct. 28, 2016 (KR) 10-2016-0141735

(51) Int. Cl. A61B 17/06

(52) U.S. Cl. CPC A61B 17/06066; A61B 2017/06052; A61B 2017/0609

(57) ABSTRACT

Provided is a surgical needle apparatus for relocating orbital fat. The apparatus includes a body part having a circular-arc shape, sharp needle parts formed on both ends of the body part, and a thread connecting part formed on a side of the body part, thread being connected to the thread connecting part. According to the invention, the incision of the conjunctiva in the eye is minimized to reduce scarring in the conjunctiva, it is easy to perform an internal fixing procedure, the orbital fat is sufficiently moved down and relocated into a lacrimal groove to enhance surgical effects, firm internal fixing is possible to minimize the possibility of a recurrence, the inconvenience of a patient is prevented, it is unnecessary for a patient to visit a hospital so as to remove the taping and the thread, and skin troubles or inflammations are not caused.

### CROSS-REFERENCE(S) TO RELATED APPLICATION

This application claims priority of Korean Patent Application No. 10-2016-0141735, filed on Oct. 28, 2016, in the Korean Intellectual Property Office, which is hereby incorporated by reference in its entirety.

### BACKGROUND OF THE INVENTION

### Field of the Invention

The present invention relates to a surgical needle apparatus, and more particularly, to a surgical needle apparatus for relocating orbital fat, which spreads a part of orbital fat depending on the distribution of orbital fat to relocate the orbital fat in a deep-sunken lacrimal groove under the eye.

### Description of the Related Art

In general, an orbital septum surrounding the fat of the lower eyelid becomes weak with advancing years, thus causing the fat in the orbital septum to bulge out and sag under the eye. Consequently, sides of the nose are shaded or pigmentation occurs on the skin, thus causing a dark circle so that a region under the eye looks dark, and skin sags and is wrinkled, thus causing a person to look tired. The reason why the fat is accumulated under the eye as such is known as a kind of aging phenomenon, and may frequently accompany disease, for example, thyroidism, renal failure, rhinitis, asthma or the like.

Once the orbital fat is generated, it is difficult to remove the orbital fat through massage or acupressure massage, and the orbital fat is not naturally restored to its original state, so that it is necessary to surgically eliminate the orbital fat in a plastic surgery or the like. A procedure in which wrinkles under the eye are enhanced while the orbital fat is eliminated is referred to as a lower eyelid surgery. This is problematic in that a surgical site may frequently have bruises because a region under the eyebrow is incised in a surgical operation, and a beauty line is removed, thus causing the face to look flat, and complications such as ectropion or involution may be developed.

Recently, a procedure of relocating orbital fat is conducted, in which the orbital fat is not completely removed, but unnecessary fat is taken out and some of the fat is relocated into a deep-sunken lacrimal groove depending on the distribution of the orbital fat, thus solving the problem of the dark circle and minimizing the sinking under the eye.

As the related art concerning the procedure of relocating the orbital fat, there are proposed a skin incising procedure in which the skin under the eye is incised and a conjunctiva incising procedure in which the conjunctiva in the eye is incised. Here, unless the skin sags severely, the conjunctiva incising procedure is preferred because scarring is rarely visible from the outside.

As a method of relocating and fixing the bulging orbital fat into the lacrimal groove in the conjunctiva incising procedure, there are proposed an external fixing method and an internal fixing method.

The external fixing method is performed as follows: the orbital fat is tied with the thread and is pulled out through a needle, the thread is fixed by taping, and then this state is maintained for about one week, so that the displaced fat is naturally adhered. The internal fixing method is performed as follows: the orbital fat under the peeled lacrimal groove is fixed at an inside by the thread.

The external fixing method is advantageous in that it is easy to perform the procedure through the conjunctiva, and the orbital fat is sufficiently moved down to a peeled space. However, the external fixing method is problematic in that the taping is required, a patient should visit a hospital within one week after the procedure so as to remove the thread, and skin troubles or inflammation may be caused by the taping or the thread passing through the skin.

The internal fixing method is advantageous in that it is more robust than the external fixing method, the possibility of a recurrence is low, and it is unnecessary for a patient to visit a hospital so as to remove the thread and taping. However, the internal fixing method is problematic in that a field of vision is very narrow at the time of incising the conjunctiva, so that it is complicated to perform an internal fixing operation, and an incision line should be long so as to secure the field of vision, thus causing serious scarring in the conjunctiva, adherence may occur, and the orbital fat is insufficiently moved down to the lacrimal groove due to a narrow space for the surgery, so that surgical effects may be lowered.

### SUMMARY OF THE INVENTION

Accordingly, the present invention has been made keeping in mind the above problems occurring in the related art, and an aspect of the present invention is directed to a surgical needle apparatus for relocating orbital fat, which has a circular-arc shape and includes needles on both ends thereof, so that the incision of the conjunctiva in the eye is minimized to reduce scarring in the conjunctiva, it is easy to perform an internal fixing procedure, the orbital fat is sufficiently moved down and relocated into a lacrimal groove to enhance surgical effects, firm internal fixing is possible to minimize the possibility of a recurrence, the inconvenience of a patient due to taping is prevented, it is unnecessary for a patient to visit a hospital so as to remove taping and thread, and skin troubles or inflammations are not caused by the taping and the thread passing through the skin.

In order to accomplish the object, the present invention provides a surgical needle apparatus for relocating orbital fat, used to fix the orbital fat that is relocated into a lacrimal groove in a surgical operation for relocating the orbital fat, the apparatus including a body part having a shape of a circular arc; sharp needle parts formed on both ends of the body part; and a thread connecting part formed on a side of the body part, thread being connected to the thread connecting part.

Further, the body part may be configured such that a first side and a second side thereof may be symmetrical with respect to each other, and the thread connecting part may be formed on a second part of the first side when the first side may be divided into three equal parts.

Further, a hole may be formed through the thread connecting part, so that the thread may be connected to the hole.

Furthermore, in a state where the needle apparatus may be inserted through an incised conjunctiva inside the eye and the needle apparatus may be connected with the orbital fat via the thread, the second side may pass through a skin tissue of the lacrimal groove to expose it to an outside, and the needle apparatus may be moved or rotated, and then the needle apparatus may be pulled out from the skin tissue through the first side, so that the thread may be locked into the skin, and a part of the orbital fat may be pulled downwards to be relocated in the lacrimal groove and the thread may be knotted, so that the relocated orbital fat may be fixed by the thread locked into the skin tissue.

According to the present invention, the surgical needle apparatus for relocating orbital fat has a circular-arc shape and includes needles on both ends thereof, so that the incision of the conjunctiva in the eye is minimized to reduce scarring in the conjunctiva, it is easy to perform an internal fixing procedure, the orbital fat is sufficiently moved down and relocated into a lacrimal groove to enhance surgical effects, firm internal fixing is possible to minimize the possibility of a recurrence, the inconvenience of a patient due to taping is prevented, it is unnecessary for a patient to visit a hospital so as to remove taping and thread, and skin troubles or inflammations are not caused by the taping and the thread passing through the skin.

### BRIEF DESCRIPTION OF THE DRAWINGS

The above and other aspects, features and advantages of certain exemplary embodiments of the present invention will be more apparent from the following description taken in conjunction with the accompanying drawings, in which:

FIG. 1 is a view illustrating a surgical procedure when orbital fat is relocated by a conventional internal incision method;

FIGS. 2A and 2B are perspective views illustrating a needle apparatus according to an embodiment of the present invention;

FIG. 3 is a front view illustrating the needle apparatus according to the embodiment of the present invention;

FIGS. 4A to 4D are views illustrating a surgical procedure for relocating orbital fat using a needle apparatus according to an embodiment of the present invention;

FIGS. 5A to 5F are views illustrating a detailed process of fixing thread of the needle apparatus according to the embodiment of the present invention to the skin;

FIG. 6 is a view illustrating a state in which thread is connected to the needle apparatus according to the embodiment of the present invention; and

FIG. 7 is a view illustrating an outcome after surgery for relocating orbital fat using to the needle apparatus according to the embodiment of the present invention.

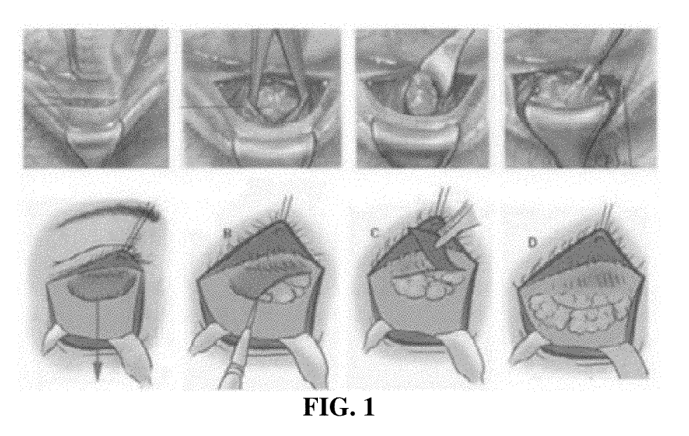

### DETAILED DESCRIPTION OF EXEMPLARY EMBODIMENTS

Exemplary embodiments of the present invention will be described below in detail with reference to the accompanying drawings.

A surgical needle apparatus 10 for relocating orbital fat according to the present invention is used to fix orbital fat when a part of the orbital fat is spread depending on the distribution of the orbital fat and is relocated in a deep-sunken lacrimal groove under the eye. The surgical needle apparatus 10 includes a body part 100, needle parts 200, and a thread connecting part 300.

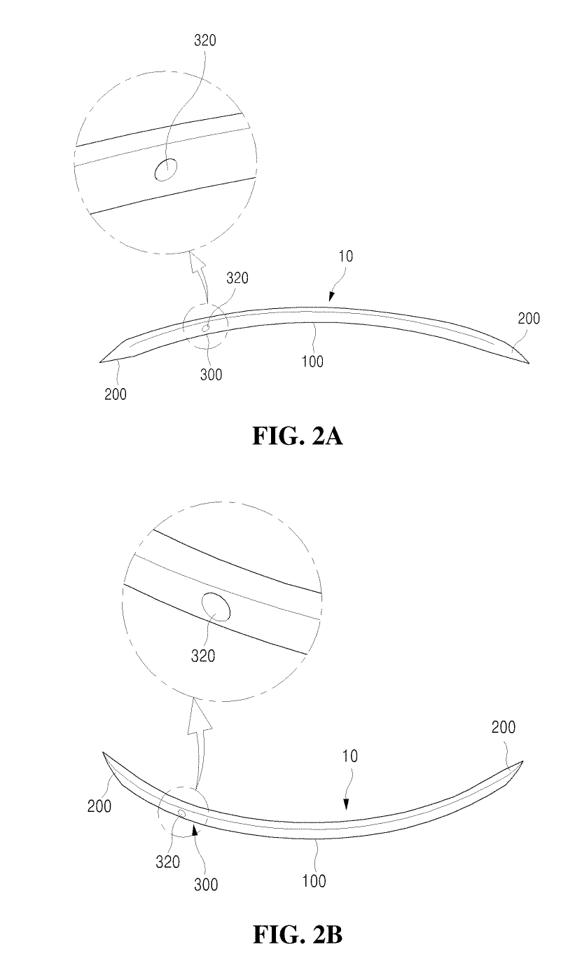

Referring to FIGS. 2A and 2B, preferably, the body part 100 is in the shape of a circular arc and has a size corresponding to ¾ of a half circle.

The needle parts 200 are sharply formed on both ends of the body part 100 to function as a needle and thereby penetrate into the orbital fat or skin and pull out the fat. Since the needle parts 200 are formed on the both ends of the body part 100, unlike the related art in which the needle part is formed only on one end, it is easy to perform an internal fixing procedure for the orbital fat even in a limited field of vision when accessing the interior through the conjunctiva, the incision of the conjunctiva is minimized, the reliability of fixing the orbital fat at a lower position is increased, and a high fixing force is guaranteed.

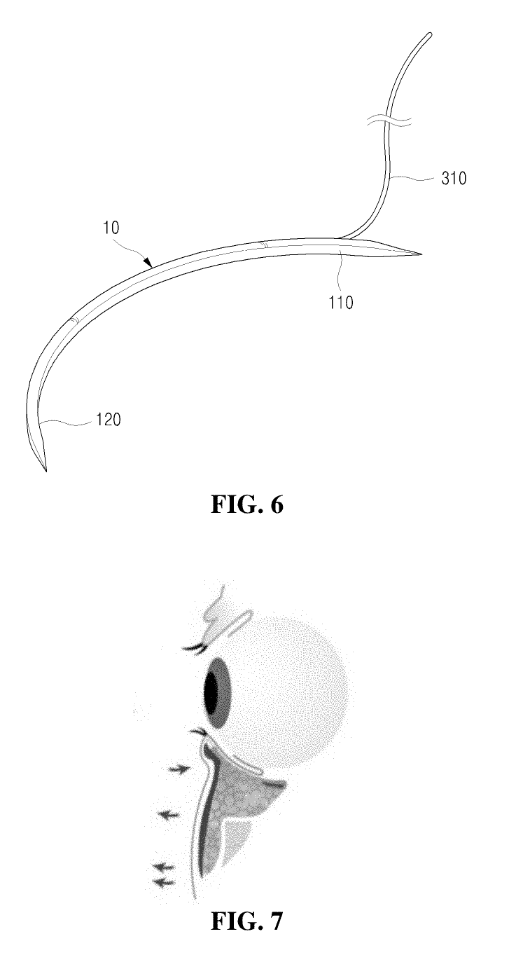

The thread connecting part 300 is formed only on one side of the body part 100 and is not formed on the other side, and thread 310 is connected to the thread connecting part 300. For example, a hole 320 is formed through the thread connecting part 300. The thread 310 may be connected to the hole 320. Alternatively, the thread 310 may be simply attached to the thread connecting part 300, as illustrated in FIG. 6.

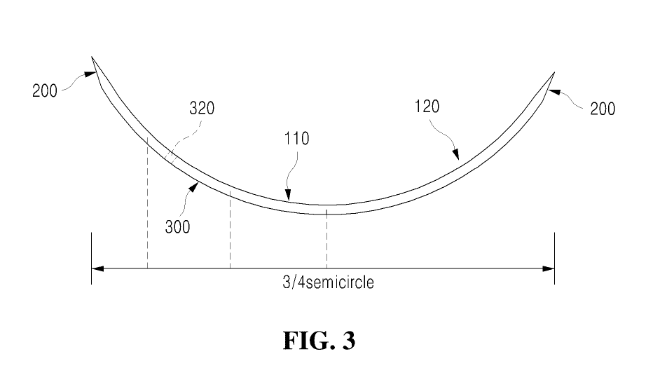

Referring to FIG. 3, the body part 100 is configured such that a first side 110 and a second side 120 thereof are symmetrical with respect to a center, and the thread connecting part 300 is formed on the first side 110.

In this connection, when the first side 110 is divided into three equal parts, the thread connecting part 300 may be formed on a second part. That is, the thread connecting part 300 is preferably on a middle portion of the first side 110 between a ⅓ portion and a ⅔ portion of the first side 110. If the thread connecting part 300 is formed too near to the needle part 200 that is located at one end, it may be difficult to fix the thread into a skin tissue during the procedure. In contrast, if the thread connecting part 300 is formed too distant from the needle part 200, the skin may be damaged or dimples, namely, uneven skin surfaces may be generated after the procedure.

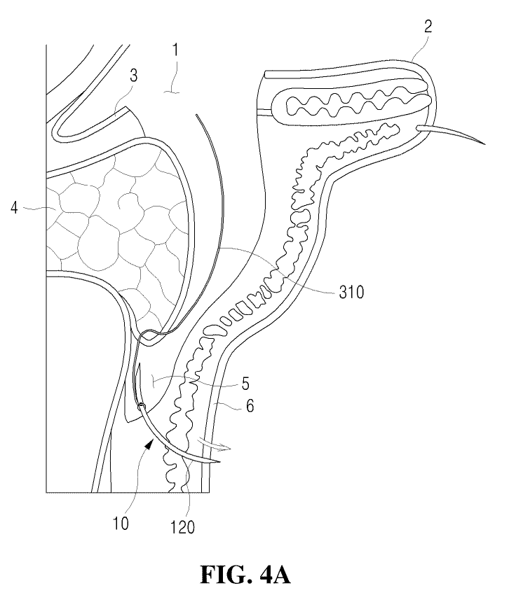

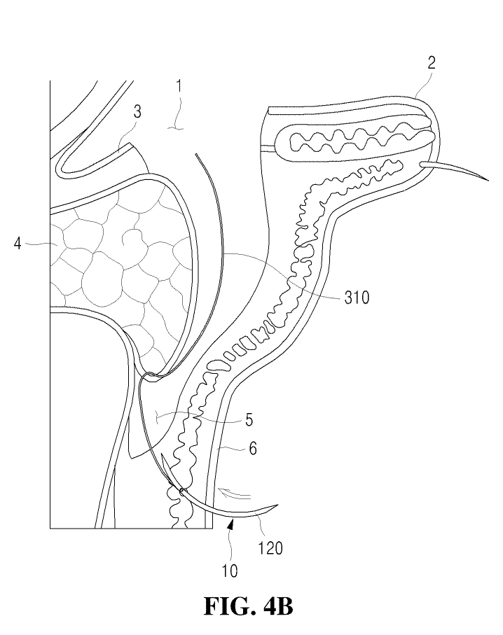

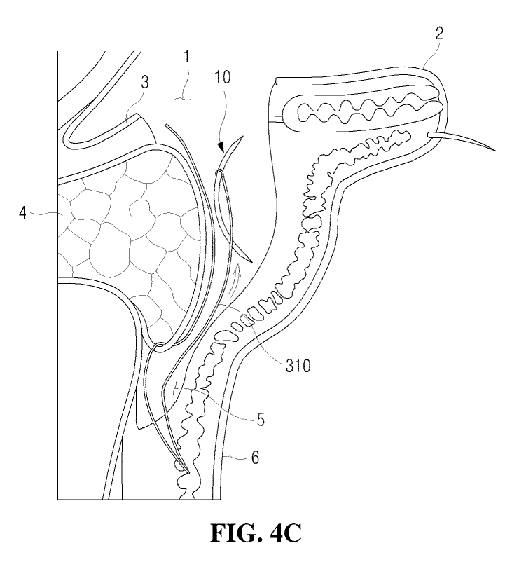

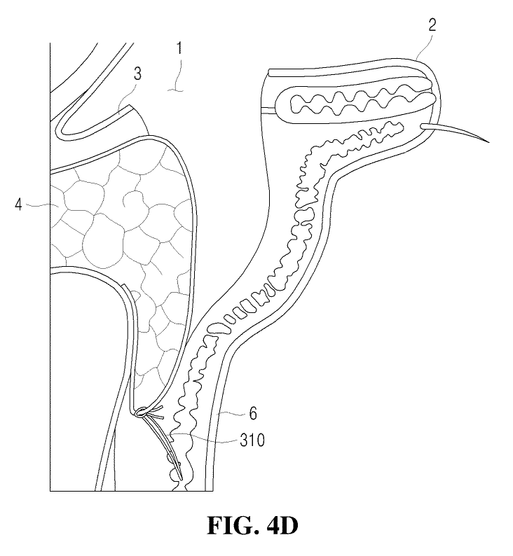

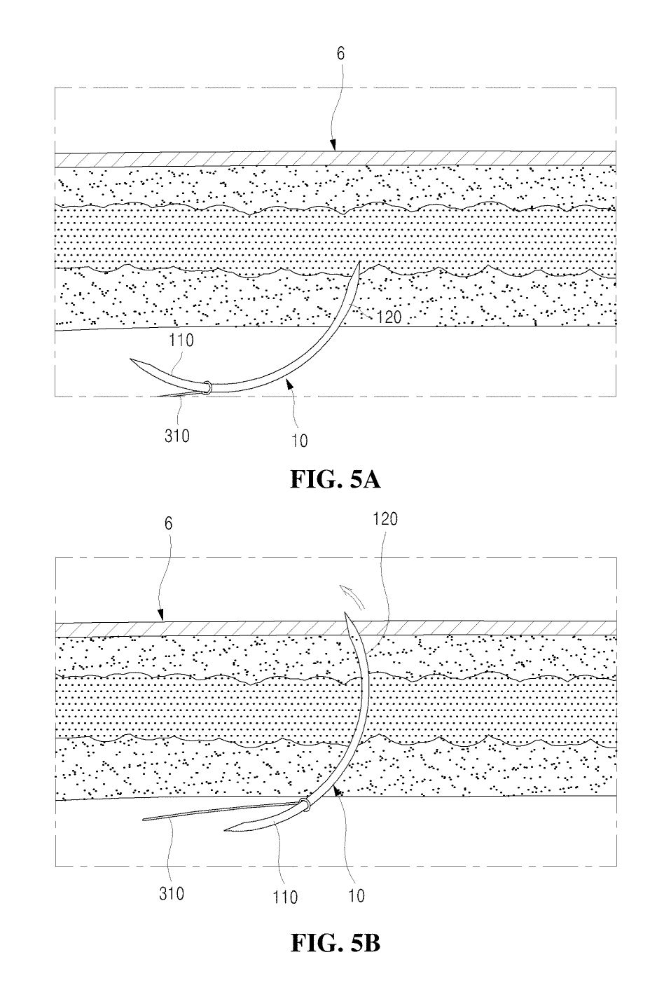

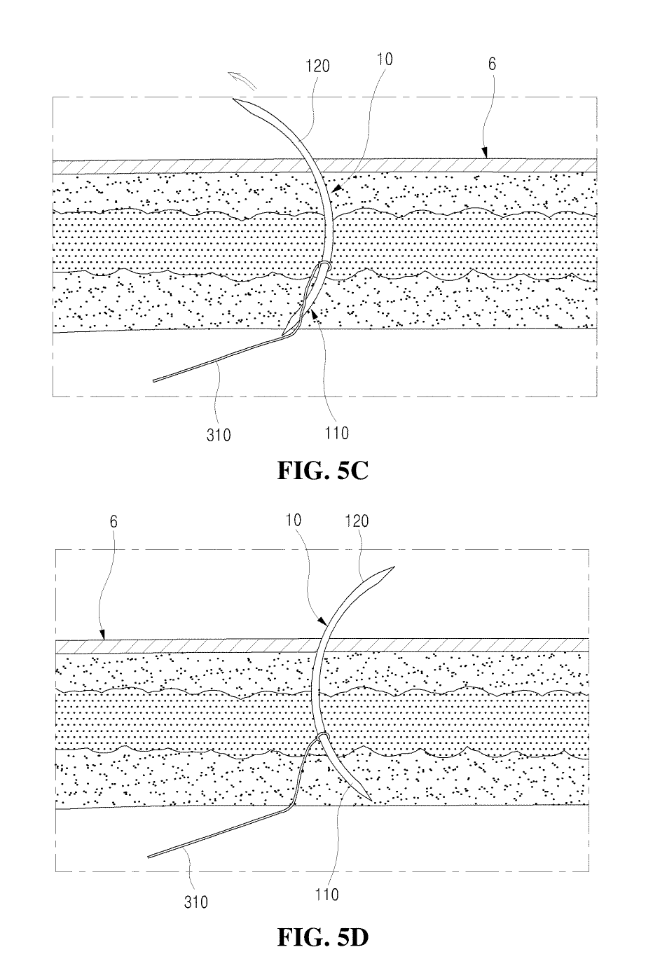

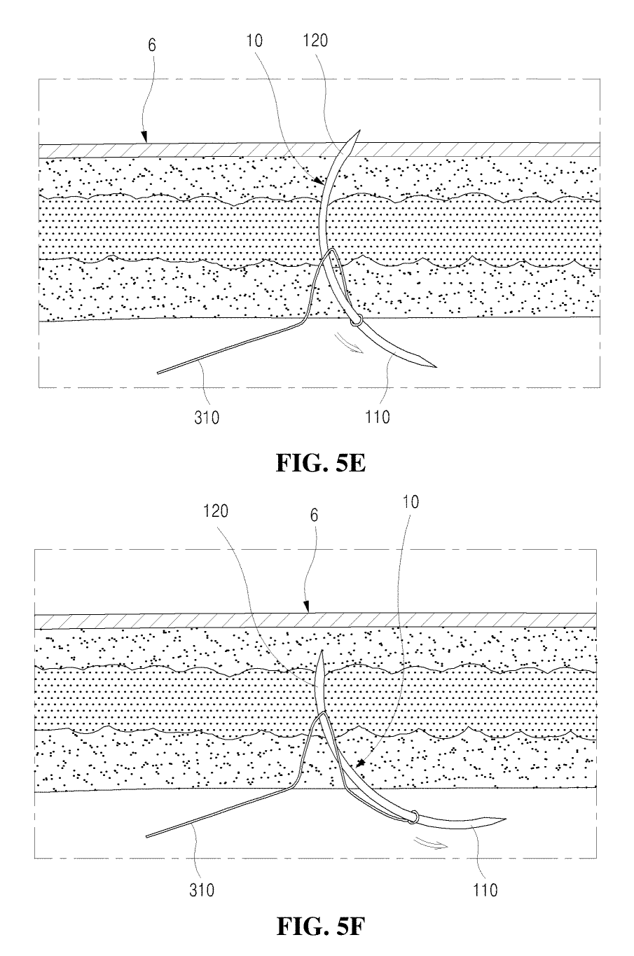

Hereinafter, the surgical procedure using the needle apparatus 1 according to the present invention will be described with reference to FIGS. 4A to 5F.

The conjunctiva 3 inside the lower eyelid 2 is incised to the minimum by a radio knife or laser, and the needle apparatus 10 and the orbital fat 4 are connected by the thread while the needle apparatus 10 of the present invention is inserted through the incised conjunctiva 3 as illustrated in FIG. 4A. When the needle apparatus 10 reaches the lacrimal groove 5 under the orbit 1, the second side 120 of the needle apparatus 10 passes through the skin tissue 6 outside the lacrimal groove 5 to expose it to an outside, and the first side 110 penetrates into the skin tissue 6 together with the thread 310.

Here, after the needle apparatus 10 is rotated or moved at the outside as illustrated in FIG. 4B, the needle apparatus 10 is drawn out from the skin tissue 6 through the first side 110 and then is pulled upwards as illustrated in FIG. 4C, so that the thread 310 is locked into the skin tissue. This is possible because the body part 100 has the shape of the circular arc and the sharp needle parts 200 are formed on the both ends of the body part 100.

By way of example, this process will be described in detail. The second side 120 of the needle apparatus 10 is penetrated into the skin tissue 6 as illustrated in FIGS. 5A and 5B and the second side 120 is partially or entirely pulled out of the skin tissue 6 as illustrated in FIG. 5C. Subsequently, if the needle apparatus 10 is pushed inwards as illustrated in FIG. 5E in the state where the needle apparatus 10 is rotated in an opposite direction at the outside as illustrated in FIG. 5D, the thread 310 is locked into the skin tissue 6 as illustrated in FIG. 5F.

Thereafter, if a part of the orbital fat 4 is pulled downwards to be relocated in the deep-sunken lacrimal groove 5 and the thread 310 is knotted, the relocated orbital fat 4 is fixed by the thread 310 that is locked into the skin tissue 6. Consequently, this makes it easy to perform the internal fixing procedure of the orbital fat without causing bleeding or bruises, allows the orbital fat to be sufficiently moved down and relocated into the lacrimal groove, thus enhancing the surgical effect, and has high fixing force, thus preventing a recurrence.

Thus, when the surgical operation has been completed using the needle apparatus 1 according to the present invention, as illustrated in FIG. 7, a dark circle does not occur, the orbital fat is relocated to increase a volume under the eye throughout a sufficient area from the deep-sunken lacrimal groove to cheekbones, thus achieving a natural young face.

As described above, the present invention provides a surgical needle apparatus 1 for relocating orbital fat, which has a circular-arc shape and includes needles on both ends thereof, so that the incision of the conjunctiva in the eye is minimized to reduce scarring in the conjunctiva, it is easy to perform an internal fixing procedure, the orbital fat is sufficiently moved down and relocated into a lacrimal groove to enhance the surgical effect, firm internal fixing is possible to minimize the possibility of a recurrence, the inconvenience of a patient due to taping is prevented, it is unnecessary for a patient to visit a hospital so as to remove the taping and the thread, and skin troubles or inflammations are not caused by the taping and the thread passing through the skin.

While the invention has been shown and described with reference to exemplary embodiments thereof, it will be understood by those skilled in the art that various changes in form and details may be made therein without departing from the spirit and scope of the invention as defined by the appended claims.

Therefore, the scope of the invention is defined not by the detailed description of the invention but by the appended claims, and all differences within the scope will be construed as being included in the present invention.

What is claimed is:

1. A surgical needle apparatus for relocating orbital fat used to fix the orbital fat that is relocated into a lacrimal groove in a surgical operation for relocating the orbital fat, the apparatus comprising:
a body part having a shape of a circular arc;
sharp needle parts formed on both ends of the body part; and
a thread connecting part formed on a side of the body part, thread being connected to the thread connecting part.

2. The surgical needle apparatus of claim 1, wherein the body part is configured such that a first side and a second side thereof are symmetrical with respect to each other, and
the thread connecting part is formed on a second part of the first side when the first side is divided into three equal parts.

3. The surgical needle apparatus of claim 1, wherein a hole is formed through the thread connecting part, so that the thread is connected to the hole.

4. The surgical needle apparatus of claim 3, wherein, in a state where the needle apparatus is inserted through an incised conjunctiva inside the eye and the needle apparatus is connected with the orbital fat via the thread, the second side passes through a skin tissue of the lacrimal groove to expose it to an outside, and the needle apparatus is moved or rotated, and then the needle apparatus is pulled out from the skin through the first side, so that the thread is locked into the skin, and
a part of the orbital fat is pulled downwards to be relocated in the lacrimal groove and the thread is knotted, so that the relocated orbital fat is fixed by the thread locked into the skin tissue.
